(19) United States
(12) Patent Application Publication
(10) Pub. No.: US 2011/0159480 A1
(43) Pub. Date: Jun. 30, 2011

(54) CELL-BASED SCREENING ASSAY TO IDENTIFY MOLECULES THAT STIMULATE IFN-ALPHA/BETA TARGET GENES

(75) Inventors: Pierre-Olivier Vidalain, Puteaux (FR); Frederic Tangy, Les Lilas (FR); Helene Munier-Lehmann, Meudon (FR); Alexandru Lupan, Boulogne Billancourt (FR); Marianne Lucas-Hourani, Issy-les-Moulineaux (FR); Yves Jacob, Maintenon (FR)

(73) Assignee: INSTITUT PASTEUR, Paris (FR)

(21) Appl. No.: 12/648,225

(22) Filed: Dec. 28, 2009

(51) Int. Cl. C12Q 1/68

(52) U.S. Cl. 435/6

(57) ABSTRACT

A method using cells transformed with a construct containing a reporter gene operatively-linked to an interferon stimulated response element for identifying an agent, such as a chemical compound, peptide or polypeptide, which exhibits an interferon-like activity on the interferon stimulated response element (ISRE) and modulates genes activated by activation of the ISRE.

### BACKGROUND OF THE INVENTION

1. Field of the Invention

A method using cells transformed with a construct containing a reporter gene operatively-linked to an interferon stimulated response element for identifying an agent, such as a chemical compound, peptide or polypeptide, which exhibits an interferon-like activity on the interferon stimulated response element (ISRE) and modulates genes activated by activation of the ISRE. The method may be used to identify an agent that activates an ISRE, but lacks other undesirable activities associated with interferon, such as therapeutic side-effects, or used to identify an agent which avoids the blocking effects of interferon inhibitors, such as certain viral proteins.

2. Description of the Related Art

Interferons α and β (IFN-α/β) are essential mediators of the antiviral immune response. They determine the establishment of a resistance state through the induction of a gene cluster encoding proteins and RNA molecules with potent antiviral activities (Stark et al., 1998). For this reason, recombinant IFN-α/β have been used with success in the treatment of chronic hepatitis B and C. However, these molecules have significant side effects, such as flu-like syndromes, dizziness and depression, and were disappointing in the treatment of most viral infections and virus-induced cancers. The large panel of molecular mechanisms that viruses evolved to block IFN-α/β signaling is probably the main reason to explain this setback (Randall and Goodbourn, 2008).

Indeed, viruses have developed various strategies to block both IFN-α/β induction and IFN-α/β signaling to prevent the induction of IFN-stimulated genes. Thus, chemical compounds restoring the expression of IFN-α/β target genes in virus infected cells with minimal side effects would be of major clinical interest in the treatment of viral infections and virus-induced cancers.

Interferons α and β (IFN-α/β) are essential mediators of the antiviral immune response, and many reports have established the critical need for viruses to block this pathway to replicate. As a consequence, viruses have evolved various strategies to escape the antiviral activity of IFN-α/β, and prevent the induction of IFN-stimulated genes. For example, the V protein of measles virus interferes with IFN-α/β signaling through interaction with STAT1, STAT2 and Jak1. IFN-α/β are induced by a large panel of cellular receptors like Toll-like receptors, RIG-I and MDA5 that recognize pathogen-associated molecular patterns such as naked RNA molecules or viral glycoproteins. They signal through direct binding to a cell surface receptor composed of two transmembrane subunits, IFNAR1 and IFNAR2c. To transduce signals, this receptor relies on two members of the Janus tyrosine kinase (JAK) family, Tyk2 and Jak1, which constitutively bind IFNAR1 and IFNAR2c, respectively. Two members of the signal transducer and activator of transcription (STAT) family, STAT1 and STAT2, are also essential components of this pathway. Upon binding of IFN-α/β to its receptor, activated Jak1 and Tyk2 phosphorylate STAT1 and STAT2. Together with the STAT-associated factor IRF9, STAT1 and STAT2 assemble into a heterotrimeric complex known as the IFN-stimulated gene factor 3, ISGF3. ISGF3 accumulates in the nucleus where it binds conserved IFN-stimulated response element (ISRE) sequences, and stimulates the expression of an antiviral gene cluster. In addition to this canonical pathway, some cellular proteins such as IRF3/7 (Ning S. & al., J.B.C., 2005) and chemicals like ethanol (Plumlee C. R., Virology J., 2005) have been reported to activate the transcription of ISRE-controlled genes.

### BRIEF SUMMARY OF THE INVENTION

“Cell” refers to cells which can be transformed and express a construct comprising the ISRE and reporter gene. Generally, this will refer to mammalian cells, including human cells and other conventional mammalian cell lines including murine cell lines. However, insect, amphibian, reptilian, yeast, and prokaryotic cell lines may be also used so long as they can be transformed and can express the ISRE-reporter gene construct. Such cell lines may be established cell lines, immortalized cell lines or transformed cell lines.

“Interferon stimulated response element” or “ISRE” is a polynucleotide sequence which is activated by contact with an interferon, such as α/β-interferon. Such ISREs are well-known in the art. and are also incorporated by reference to Hug, et al., Mol. Cell. Biol. 8(8): 3065-3079 (1988); and Darnell, et al., Science 264: 1415-1421 (1994). In general, an ISRE sequence will contain the consensus sequence N(0-2)GAAAN(1-2)GAAAN(0-2) (SEQ ID NO: 1) (wherein N is present 0, 1 or 2 times as indicated in parenthesis) or its reverse complement N(0-2)CTTTN(1-2)CTTTN(0-2) (SEQ ID NO: 2) (wherein N is present 0, 1 or 2 times as indicated in parenthesis).

(SEQ ID NO: 2). Accordingly, an ISRE may be described as a regulatory sequence that complies with the following two conditions:

(i) it has a sequence that matches the consensus N(0-2)GAAAN(1-2)GAAAN(0-2) (SEQ ID NO: 1) where N equals any nucleotide and the numbers represent the presence of 0, 1, or 2 of the preceding nucleotides (or matches the reverse complement of this sequence) and

(ii) is activated in cells responsive to IFN-α/β when stimulated with IFN-α/β.

The following non-exhaustive list of ISREs are incorporated by reference to Collet, et al., Eur. J. Biochem. (2004) Volume 268 Issue 6, Pages 1577-1584 and Geiss, et al., J. Virol. 77(11): 6367-6375 (2003). Such elements include, but are not limited to, those depicted in the tables reproduced from these two references below:

TABLE 1
ISRE sequences

| Gene | SEQ ID NO: | Sequence |
|---|---|---|
| ABCB2 | 5 | GAAAGCGAAAGC |
| ABCB2 | 6 | GAAATCGAAAGC |
| ABCB2 | 7 | GAAAGCGAAATC |
| ADAR | 8 | GAAACGAAAGC |
| ADAR | 9 | GAAAGCGAAATT |
| CASP1 | 10 | GAAATAGAAAC |
| CASP1 | 11 | GAAACTGAAAGT |
| G1P3 | 12 | GAAAAAGAAAC |
| G1P3 | 13 | GAAAATGAAACT |
| G1P3 | 14 | GAAAATGAAACT |
| G1P3 | 15 | GAAATAGAAAC |
| GBP1 | 16 | GAAACTGAAAGT |
| GS3686 | 17 | GAAATGAAAGC |
| IFI16 | 18 | GAAACGAAAGC |
| IFIT1 | 19 | GAAACTGAAAAT |
| IFIT1 | 20 | GAAAGTGAAACT |
| INP10 | 21 | GAAAGTGAAACC |
| ISGF3 | 22 | GAAAGGAAAC |
| KIAA1268 | 23 | GAAACGAAAGC |
| MX1 | 24 | GAAAGAGAAAC |
| MX1 | 25 | GAAACGAAAC |
| MX1 | 26 | GAAATGAAAC |
| MYD88 | 27 | GAAAGCGAAAGC |
| NMI | 28 | GAAAGTGAAATT |
| PRKR | 29 | GAAAACGAAACT |
| PRKR | 30 | GAAAGGAAAC |
| PRKR | 31 | GAAAGGAAAC |
| PSME2 | 32 | GAAAGCGAAAGC |
| RI58 | 33 | GAAACCGAAACT |
| RI58 | 34 | GAAATCGAAACT |
| RI58 | 35 | GAAACTGAAACT |
| RNF21 | 36 | GAAAGTGAAATT |
| STAT1 | 37 | GAAAGCGAAACT |
| TNFSF10 | 38 | GAAATGAAAGC |
| Consensus 1 | 39 | GAAANGAAAGC |
| Consensus 2 | 40 | GAAANNGAAAGC |

SEQ ID NOS: 5-40 are described by Geiss, et al., J. Virol. 77(11): 6367-6375 (2003) and additional information about these sequences and methods useful for structural and functional characterization of ISRE sequences are specifically incorporated by reference to Geiss, et al.

TABLE 2
Mammalian ISREs and Consensus ISRE sequences

| | SEQ | |
|---|---|---|
| | ID | |
| | NO: | |
| Human ISREs | | |
| As HLA-A3 | 41 | A GAAA A - GAAA C T |
| S 2′,5′AS(−99/−88) | 42 | G GAAA C - GAAA C C |
| As 2′,5′AS p69 | 43 | G GAAA C T GAAA C T |
| S 6-16(−152/−140) | 44 | G GAAA A T GAAA C T |
| S 6-16(−111/−99) | 45 | G GAAA C T GAAA C T |
| S Isg20(−49/−39) | 46 | A GAAA C T GAAA C A |
| as 56 kDa(−119/−107) | 47 | G GAAA G T GAAA C T |
| sISG15 (−107/−95) | 48 | G GAAA C C GAAA C T |
| as ISG54 (−103/−91) | 49 | G GAAA G T GAAA C C |
| as factor B (−140/−128) | 50 | G GAAA C A GAAA C T |
| s IFN-α1 (−85/−73) | 51 | A GAAA T G GAAA G T |
| s PKR | 52 | G GAAA A C GAAA C T |
| s ADAR1 | 53 | G GAAA - CGAAA G C |
| s IP10 (−228/−221) | 54 | G GAAA C T GAAA C C |
| as MxA (−102/−91) | 55 | A GAAA - C GAAA C C |
| Murine ISREs | | |
| s PKR | 56 | G GAAA A C GAAA C A |
| s 202 (−143/−131) | 57 | G GAAA T T GAAA G C |
| as Mx (−131/−120) | 58 | A GAAA C - GAAA C T |
| Chicken ISRE | | |
| as Mx (−61/−50) | 59 | A GAAA C - GAAA C T |
| Rainbow Trout ISREs | | |
| as Vig-2 | 60 | T GAAA G T GAAA C T |
| s Mx2 (−101/−88) | 61 | T GAAA G T GAAA C A |
| Consensus ISRE | | |
| Friedman-Stark | 62 | GAAA C T |
| Hug (first motif) | 63 | R GAAA N - GAAA S Y |
| Hug (second motif) | 64 | R GAAA N N GAAA S Y |
| ISRE | 65 | N GAAA N N GAAA C T |
| Revised Consensus (1) | 66 | D GAAA N - GAAA S H |
| Revised Consensus (2) | 67 | D GAAA N N GAAA S H |

In the above table “-” indicates that there is no amino acid residue at that relative position, and is shown to indicate the alignment of residues amongst different sequences. “R” indicates purine; “Y” indicates pyrimidine; “S” indicates “G” or “C”; “H” refers to “A”, “C” or “T”; “D” indicates “A”, “G” or “T”; and “N” indicates any nucleotide base (e.g., “A”, “C”, “G” or “C”). Ribonucleic acid equivalents of these sequences are also contemplated, as are modified polynucleotide sequences having modified backbones or equivalent derivatized bases. Any of the above sequences may be made synthetically. Materials and methods for structurally and functionally characterizing ISREs are incorporated by reference to Collet, et al., Eur. J. Biochem. 268: 1577-84 (2004), which also discloses the sequences shown by SEQ ID NOS: 41-68 in the table above. ISRE sequences and consensus motifs for ISREs are also specifically incorporated by reference to Collet, et al.

A “reporter gene” or “reporter” is a polynucleotide which is attached to a polynucleotide of interest and may be expressed within a cell to confer a particular phenotype which is easily measured or identified or which is or encodes a selectable marker. Luciferase is one type of reporter gene used in the invention, however, any suitable reporter gene may be operatively-linked to an ISRE. Such reporter genes include those which encode green fluorescent protein (GFP), dsRed, mCherry, Yellow Fluorescent protein firefly luciferases, beta-galactosidase, beta-lactamase and secreted alkaline phosphatase and are specifically incorporated by reference to Current Protocols in Molecular Biology, Chapter 9 (2008), especially sections 9.6-9.8.

“Agent” includes atoms, ions, molecules, compounds, and polymers. Generally such agents will be organic molecules, drugs, pharmaceutical compounds, polymeric biomolecules such as peptides, proteins or nucleic acids. This term also includes chemical variants of such molecules, such as chemically modified peptides or oligonucleotides. Agents include peptides or polypeptides fused to cell-penetrating sequences can cross membranes to activate ISRE's. Such peptides or polypeptides don't need to be expressed within cells. For example, the cells may be incubated in the presence of these peptides. Other organic or peptidic substances may also be used to force peptides to enter the cell and such methods are incorporated by reference to Howl, et al., Biochem. Soc. Trans. 35(Pt 4):767-9 (2007). For example, a polypeptide or peptide may be PEGylated by methods well-known in the art and its pharmacological properties modified, see Veronese, et al., Drug Discovery Today 10(21): 1451-1458 (2005) which is hereby incorporated by reference.

“Interferon α/β” refers to interferon α or interferon β. The protein sequences for these interferons and the polynucleotide sequences encoding them are well-known in the art and are specifically incorporated by reference to Pestka, J.B.C. 282 (28):20047-51 (2007) and to the documents or databases cited by Pestka as disclosing such sequences.

Specific embodiments of the invention include, but are not limited to the following.

A method for identifying an agent that exhibits an interferon-like activity comprising contacting a test agent with a cell that expresses reporter construct comprising at least one IFN-stimulated response element (ISRE) operatively linked to a reporter gene, and detecting an agent that activates the reporter gene, wherein activation of the reporter gene is indicative of an agent that exhibits an interferon-like activity. The agents, including molecules and compounds, to be screened may include peptide, such as peptide fragments of 2-20 amino acid residues, or polypeptides, including chemically modified polypeptides or fragments of a type I interferon. Oligonucleotides or nucleic acid molecules may also be screened, as well as molecules that are not peptides, polypeptides, or nucleic acids, such as small organic molecules having a molecular weights ranging from 50 to 2,500 Da.

Cells used in the methods are preferably mammalian cells, including human cells, and may include cells from established, transformed or immortalized cell lines. Non-mammalian cells may also be used if they contain genes responsive to activation by Interferon stimulated response gene element.

The cell line will contain or be transformed with, a reporter construct comprising at least one IFN-stimulated response element (ISRE) operatively linked to at least one reporter gene. Optionally, other elements may be transformed or expressed by the cell line, such as interferons or viral components. The ISRE may be derived from a non-human mammal or from a human. Representative reporter constructs include the pISRE-luc plasmid which has been deposited on Jul. 31, 2008 under the number 1-4060 under the terms of the Budapest Treaty at the Collection Nationale de Culture de Microorganismes (CNCM), Institut Pasteur 25 rue du Docteur Roux F-75724 PARIS Cedex 15.

Various methods for screening cells for reporter gene expression are well known in the art and are also incorporated by reference to Current Protocols in Molecular Biology, Chapter 9 (2008). Screening methods include high-throughput methods as illustrated in FIG. 2.

The method of the invention is based on measuring the ISRE-driven reporter gene expression in said cell, may comprise the identification of a molecule that induces IFN-α/β expression in said cell, or alternatively may involve detecting molecules that activate the ISRE regulated reporter gene construct in the absence of or independently of IFN-α/β molecule(s), thus identifying an IFN-α/β independent molecule.

### BRIEF DESCRIPTION OF THE DRAWINGS

FIG. 1. HEK-293T cells were transfected with pISRE-Luc, pRL-CMV, and pEGFP-C1 expression plasmids encoding indicated proteins. 24 h after transfection, 1000 IU/ml of recombinant IFN-β were added. After 24 h of incubation, the relative luciferase activities of IFN-treated cells were determined. This result illustrates the induction of a luciferase reporter gene under control of an ISRE element when stimulated by IFN-α/β. Measles virus V protein, and to a lower extend the Y110 mutant that fail to interact with STAT1, block the IFN-α/β pathway.

FIG. 2. High-throughput screening protocol. (1) Organic compounds diluted in DMSO were disposed in the wells of a white flat-bottom 96-well plate. (2) HEK-293T cells in suspension were transfected with an ISRE-luciferase reporter plasmid complexed with poly-ethylenimine (PEI) that is used as a transfection reagent. (3) 100 μL of the transfected cell suspension were disposed in each wells of the plate, and incubated for 24 h at 37° C. and 5% CO₂. (4) Finally, 50 μL of Bright-Glo™ reagent (a luciferase reagent from Promega) were added to each well before reading luminescence.

FIG. 3. Activity of different compounds on the ISRE-luciferase reporter.

FIG. 4. Chemical structure of IHK105-F08.

### DETAILED DESCRIPTION OF THE INVENTION

The inventors have found that compounds stimulating the expression of interferon-stimulated genes, either by Jak/STAT or other yet unidentified pathways, exist and can be identified by screening libraries. Such chemical compounds, peptides or proteins can be identified using cells expressing, either transiently or constitutively, an ISRE-response element upstream of a screenable reporter gene like luciferase. Compounds, peptides or proteins that stimulate the IFN-α/β-stimulated gene cluster by trans-stimulation of the ISRE regulatory element are likely to exhibit some antiviral activity by stimulating the innate antiviral immune response. In addition, such molecules could overcome the inhibitory mechanisms developed by viruses to impair the IFN-α/β response. Finally, because IFN-α/β have been used with success against some cancers, compounds that activate IFN-α/β-stimulated genes could be of interest in oncology.

The present invention describes a system to identify chemical compounds, peptides or proteins that stimulate the expression of IFN-α/β target genes or that exhibit some antiviral activity by stimulating the cluster of antiviral genes activated by type I interferons (Geiss et al., 2003). This system is based on human cells transfected, transiently or stably, with a reporter construct composed of a luciferase reporter gene fused downstream of an IFN-stimulated response element (ISRE). This regulatory sequence is found in the promoter sequence of most IFN-α/β target genes. Upon IFN-α/β stimulation, ISRE sequences bind a heterotrimeric complex formed by STAT1, STAT2 and IRF9. Consequently, this assay can be used to identify molecules that stimulate the expression of IFN-α/β target genes (Geiss et al., 2003; Stark et al., 1998). Molecules identified with this assay can be separated in two categories: (i) molecules that induce IFN-α/β expression in treated cells (IFN-α/β dependent molecules), and (ii) molecules that activate ISRE regulated genes independently of the IFN-α/β loop (IFN-α/β independent molecules).

Both categories of molecules are of therapeutic interest. In the first category, imiquimod (commercialized as Aldara, 3M Pharmaceuticals) is the only drug available on the market and was recently used with success in the treatment of external genital warts (Hemmi et al., 2002). Regarding the second category, there is no molecule described in the literature with this activity. However, these molecules are probably the most promising in the treatment of viral infections because they could stimulate the expression of antiviral cellular genes when the canonical IFN-α/β signaling pathway is down, blocked by viral virulence factors.

The invention may use existing cell-based assays, but recognizes a new use for these assays where the assay is performed without α/β-interferon. Indeed, several ISRE-reporter gene systems are commercially available. For example, in the CellSensor® system from Invitrogen, an ISRE-bla construct has been introduced in the genome of HEK293T cells. The pISRE-Luc cis-reporter plasmid from Stratagene contains direct repeats of the interferon-stimulated response element (ISRE) found in the promoter of the IFI-54 gene. Such systems are normally used in the presence of IFN-α/β to identify inhibitors of this pathway. The CellSensor® assay steps and reagents are incorporated by reference to Invitrogen publications existing as of the filing date of this application, including “Validation & Assay Performance Summary” for CellSensor® ISRE-bla HEK23T Cell Line (Cat. No. K1105) dated Sep. 15, 2006. Stratagene assays and reagents are incorporated by reference to Stratagene publications existing as of the filing date of this application, such as Xu, et al., “New Reporter Plasmids for Studying Interferon-Stimulated Signal Transduction Pathways”, Biocompare 24 May 2007.

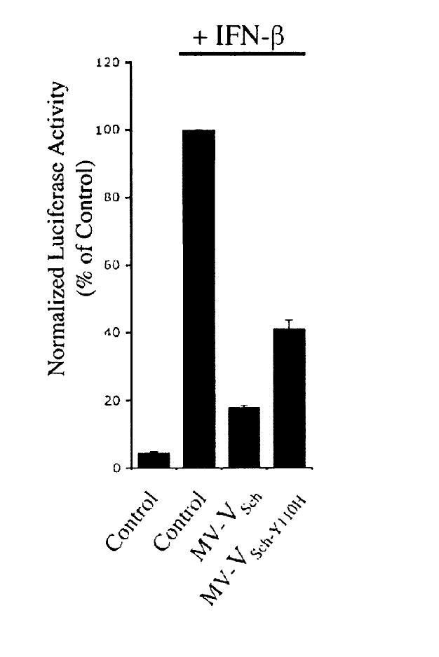

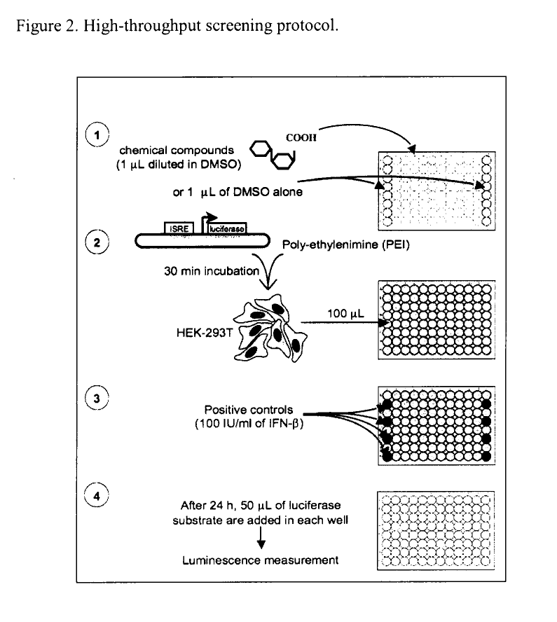

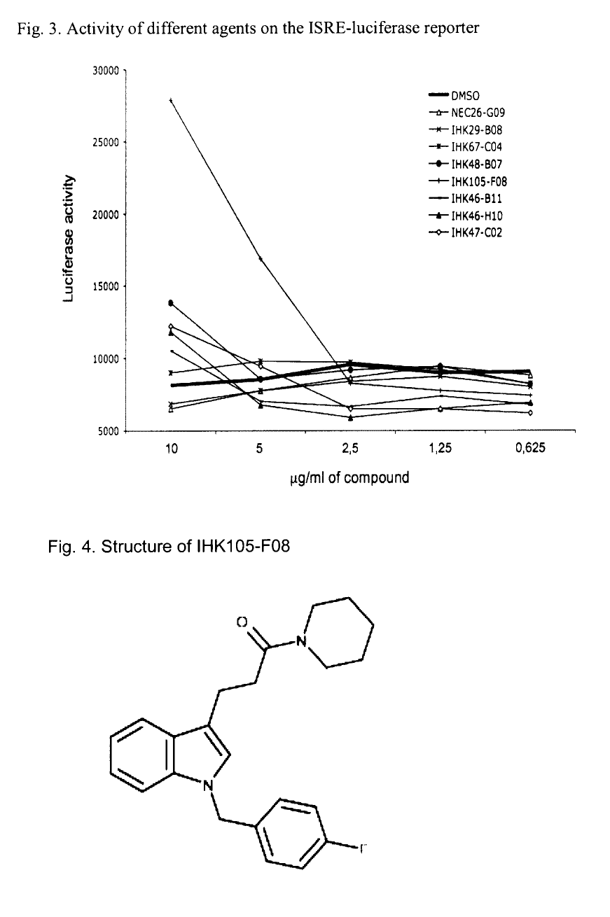

The assay described here is amenable to high-throughput, allowing the screening of large chemical compound libraries. The inventors developed a protocol to transfect HEK293T cells with the ISRE-Luc reporter construct (FIG. 1), and established a proof of concept by screening 3 chemical compound libraries. A first set of 34 molecules that induced luciferase activity above background level was selected with this screen. These molecules were retested twice using the same assay, leading to the selection one molecule identified as IHK-105-F08 (FIG. 2 and FIG. 3). As shown in FIG. 2, IHK-105-F08 stimulates the ISRE-luciferase reporter gene above background level, with an activity equivalent to 4% of recombinant IFN-β at 1,000 IU/ml. These results demonstrate that this assay is highly tractable and allows the identification of small molecules activating ISRE regulatory elements. Other suitable high-throughput assay methods may be used, such as those described by Fan, et al., Assay Drug Dev. Technol. 5(1):127-36 (2007), “Bioluminescent assays for high-throughput screening” which are hereby incorporated by reference.

The inventors have found that such assays when performed in the absence of recombinant IFN-α/β can identify ISRE activators that induce IFN-α/β target genes. Molecules identified by this method can be used in the treatment of viral infections, and because some cancers are treated with IFN-α/β injections (e.g. malignant melanoma); molecules isolated with this assay are of interest in oncology. In addition, these molecules may useful in the treatment of multiple sclerosis and autoimmune disorders because IFN-α/β exhibit some immunosuppressive activities. Molecules identified by this method may be used for similar purposes as various recombinant IFN-α/βs. However, this assay permits the identification of molecules with fewer or lessened side effects and higher activity levels than recombinant IFN-α/β products.

Once an agent that induces the ISRE response element is identified, the relative ISRE-activating activity may be further characterized by comparison to one or more control interferons and the effects of the agent on inducing of specific biological responses induced by interferons may be determined. The anti-viral or anti-tumor activity of the agent relative to one or more control interferons may be determined in vitro, ex vivo or in vivo. Similarly, whether or not the selected agent that activates the ISRE induces side-effects associated with interferon administration may be determined. For example, after or simultaneously with the identification of a test agent that activates an ISRE, the test agent may be administered to a subject, such as a mammal or human, or depending on the side-effect, in vitro or ex vivo, followed by detection of the side-effects of such administration compared to the side-effects exhibited by a control subject not administering a control α/β-interferon, but not the test agent. Side-effects may include neutropenia, lymphopenia, or other suppression or disruption of the cellular immune system or flu-like symptoms such as increased body temperature, nausea, fatigue, headache, muscle pain, convulsion, dizziness, hair thinning, depression, or other flu-like symptoms, or local reaction to administration of the test agent, such as erythema, pain or harness at a site of injection when said agent is administered by injection. The side-effects associated with IFN-α/β administration are incorporated by reference to Pestka, et al., J. Biol. Chem: 282(28):20047-51 (2007).

### EXAMPLES

### Example 1

### Effects of β-Interferon on Reporter Gene Expression in Transformed HEK-293T Cell Line and Effects of Co-Expression of Viral Proteins that Inhibit Interferon Activity on Reporter Gene Expression

HEK-293T cells were transfected with pISRE-Luc (positive control), or pISRE-Luc+pEGFP-C1-MV-V_{Sch} (MV-V_{Sch}), or pISRE-Luc+pEGFP-C1-MV-V_{Sch-Y110H} (MV-V_{Sch-Y110H}). pISRE-Luc expresses a luciferase reporter gene under the control of an ISRE element; pEGFP-C1 expresses wild-type measles virus V (MV-V_{Sch}) protein or the Y110H mutant form (MV-V_{Sch-Y110H}). Mutant Y110H contains a mutation within the PNT domain of measles virus V protein which impairs its ability to interact with and block STAT1 phosphorylation.

Transfected cells were treated with 1,000 IU/ml of recombinant IFN-β 24 hrs after transfection and incubated for 24 hrs, and then the relative luciferase activities of the IFN-treated cells were determined.

As shown in FIG. 1, the luciferase reporter gene under control of an ISRE element was stimulated by IFN-β (FIG. 1, second bar, control). Plasmid pEGFP-C1-MV-V_{Sch} encodes measles virus V protein which interferes with IFNα/β signaling through interaction with STAT1 and significantly reduced expression of the luciferase reporter gene (FIG. 1, third bar). Plasmid pEGFP-C1-MV-V_{Sch-Y110H} encodes a mutant measles virus V protein (pEGFP-C1 is from BD Biosciences Clontech). FIG. 1 shows that in the presence of either measles virus V protein that luciferase reporter gene expression induced by IFN-βwas reduced.

These results show that a screening system using an ISRE element operatively-linked to a reporter gene (in this case luciferase) can be used to measure the effects of a test agent on the target genes induced by interferon via ISRE elements.

Moreover, such a system may be used to identify agents which induce expression of the interferon target genes in the presence of viral proteins that normally would attenuate interferon target gene expression by interfering with ISRE activation. For example, the method may be performed with a cell transformed with a construct where a reporter gene is expressed under the control of an ISRE, and in the presence of a construct that expresses a protein, such as measles V, protein, that inhibits interferon responses. An agent is then selected which is not inhibited in the presence of the construct which expresses the inhibitor of interferon responses. The relative degree of inhibition or stimulation of the ISRE may be determined by comparison to an otherwise similar control cell containing the ISRE-reporter construct contacted with a control interferon instead of the agent. An α-interferon or β-interferon may be used as the control interferon, and may be matched to the source of the test cells. For example, a human interferon would be used as a control when the cells used in the screening assay are derived from humans or express human ISREs.

Similarly, a controlled comparison of the effects of an agent determined to activate the ISRE in the ISRE-reporter gene construct, may be made with a control interferon either in vitro, ex vivo or in vivo. For example, both the ISRE-activating agent and a control interferon may be administered to a test animal and the relative anti-viral, anti-tumor, or relative side-effects on the test animal compared.

### Example 2

### Screening of Chemical Compound Libraries Using HEK-293T Cells Transfected with the pISRE-Luciferase Reporter Plasmid

Three chemical compound libraries were used in this assay:

Prestwick Chemical (1120 molecules),

ChemDiv kinase inhibitors (9360 molecules) and

NECAN nucleoside analogs library (4880 molecules).

Compounds were diluted at 2 mg/ml in DMSO. Then, 1 μl of each compound was deposited in 96-well culture plates (white polystyrene, Greiner 655 083). HEK-293T cells (from ATCC) were transfected in bulk with the pISRE-luciferase reporter plasmid from (Stratagene, ISRE cis-Reporting System pISRE-Luc). For one 96-well plate, 17 μg of plasmid were diluted in 588 μl of DMEM (Gibco-Invitrogen). In parallel, 53 μg of poly(ethyleneimine) from Sigma-Aldrich (PEI) were diluted in 588 μl of DMEM (Gibco-BRL). Dilutions of pISRE-Luc and PEI were mixed together and incubated for 30 min at room temperature. This mix was added to a suspension of 2.10⁺⁶ cells in 8.85 ml of DMEM containing 10% fetal bovine serum, penicillin, and streptomycin. Finally, 100 μl of this cell suspension were added to the each well of the cell-culture plate containing chemical compounds. Cells were cultured for 24 h at 37° C. and 5% CO₂. Finally, 50 μl of Bright-Glo™ solution (Promega) were added to each well, and luminescence was measured using a luminescence reader (Safire2, TECAN). See FIG. 2.

A set of 34 molecules that induced the ISRE-luciferase reporter gene three times above background level were selected.

Molecules were retested in dose response using the same functional assay. One of these molecules (IHK105-F08) was highly reproducible in its ability to induce the ISRE-Luciferase reporter and considered as a true positive (FIGS. 3 and 4). These results demonstrate the efficacy of using cells transformed with constructs expressing a reporter gene under the control of an ISRE for identifying inducers of an ISRE response element.

The IHK 105-F08 molecule identified by the method of the invention has an activity equivalent to 4% of recombinant IFN-β at optimal dose (1,000 IU/ml)

### Modifications and Other Embodiments

Various modifications and variations of the methods disclosed above, as well as reagents, cell lines and chemical libraries as well as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the biological, chemical, immunological, medical, or pharmacological arts or related fields are intended to be within the scope of the following claims.

### INCORPORATION BY REFERENCE

Each document, patent, patent application or patent publication cited by or referred to in this disclosure is incorporated by reference in its entirety. However, no admission is made that any such reference constitutes prior art and the right to challenge the accuracy and pertinence of the cited documents is reserved.

### REFERENCES

Geiss, G. K., Carter, V. S., He, Y., Kwieciszewski, B. K., Holzman, T., Korth, M. J., Lazaro, C. A., Fausto, N., Bumgarner, R. E., and Katze, M. G. (2003). Gene expression profiling of the cellular transcriptional network regulated by alpha/beta interferon and its partial attenuation by the hepatitis C virus nonstructural 5A protein. J Virol 77(11), 6367-75.
Hemmi, H., Kaisho, T., Takeuchi, O., Sato, S., Sanjo, H., Hoshino, K., Horiuchi, T., Tomizawa, H., Takeda, K., and Akira, S. (2002). Small anti-viral compounds activate immune cells via the TLR7 MyD88-dependent signaling pathway. Nat Immunol 3(2), 196-200.
Randall, R. E., and Goodbourn, S. (2008). Interferons and viruses: an interplay between induction, signalling, antiviral responses and virus countermeasures. J Gen Virol 89(Pt 1), 1-47.
Stark, G. R., Kerr, I. M., Williams, B. R., Silverman, R. H., and Schreiber, R. D. (1998). How cells respond to interferons. Annu Rev Biochem 67, 227-64.

1. A method for identifying an agent that induces an IFN-stimulated response element (ISRE) comprising:
contacting a test agent with a cell that expresses a reporter construct comprising at least one IFN-stimulated response element (ISRE) operatively linked to a reporter gene, and
detecting an agent that results in the activation of the reporter gene,
wherein activation of the reporter gene is indicative of an agent that induces an interferon-like activity.

2. The method of claim 1, wherein said agent is a peptide or a polypeptide, which optionally may be expressed within said cell.

3. The method of claim 1, wherein said agent is a peptide fragment of a type I interferon or a chemically modified type I interferon or fragment thereof.

4. The method of claim 1, wherein said agent is not a peptide or a polypeptide.

5. The method of claim 1, wherein said agent is an oligonucleotide or a nucleic acid, which optionally may be expressed within said cell.

6. The method of claim 1, wherein said agent is a small organic molecule having a molecular weight ranging from 50 to 2,500 Da.

7. The method of claim 1, wherein said cell is a mammalian cell.

8. The method of claim 1, wherein said cell is a human cell.

9. The method of claim 1, wherein said cell is not a mammalian cell.

10. The method of claim 1, wherein said cell is from an established, transformed, or immortalized mammalian cell line.

11. The method of claim 1, wherein said ISRE comprises the consensus sequence NNGAAANNGAAANN (SEQ ID NO: 1) or its reverse complement NNTTTCNNTTTCNN (SEQ ID NO: 2).

12. The method of claim 1, wherein the ISRE is a human ISRE.

13. The method of claim 1, wherein the ISRE comprises GAAAGTGAAACT (SEQ ID NO: 3) or its reverse complement AGTTTCACTTTC (SEQ ID NO: 4).

14. The method of claim 1, wherein the reporter gene is operatively linked down-stream from the ISRE.

15. The method of claim 1, wherein the reporter gene is luciferase.

16. The method of claim 1, wherein said reporter gene is selected from the group consisting of beta-galactosidase, beta-lactamase, EGFP, YFP, and mCherry.

17. The method of claim 1, wherein the reporter construct is present on an episome.

18. The method of claim 1, wherein the reporter construct is chromosomally integrated.

19. The method of claim 1 that is a high-throughput screening method.

20. The method of claim 1, further comprising determining whether an agent which increases reporter gene expression also increases IFN-α/β expression by said cell.

21. The method of claim 1, further comprising determining whether an agent which increases reporter gene expression does not increase IFN-α/β expression by said cell, thus identifying an IFN-α/β independent agent.

22. The method of claim 1, further comprising
administering a test agent determined to increase the expression of the reporter gene to a subject and
detecting the relative number or intensity of one or more side-effects of interferon α/b when compared to those in a test subject administered a control α/β-interferon, but not the test agent.

23. The method of claim 22, wherein said side-effects detected comprise at least one of neutropenia, lymphopenia, or other suppression or disruption of the cellular immune system.

24. The method of claim 22, wherein said side effect(s) comprises at least one erythema, pain or harness at a site of injection of said test agent, increased body temperature, nausea, fatigue, headache, muscle pain, convulsion, dizziness, hair thinning, depression, or other flu-like symptoms.

25. The method of claim 1, further comprising
administering a test agent determined to increase the expression of the reporter gene to a subject and
detecting the anti-viral or anti-oncogenic effects of the test agent compared to those exhibited by a subject administered a control α/β-interferon instead of the test agent.


SEQUENCE LISTING (67 sequences)

SEQ ID NO: 1 - DNA, 14 bp, artificial
  synthetic
nngaaanngaaann

SEQ ID NO: 2 - DNA, 14 bp, artificial
  synthetic
nnctttnnctttnn

SEQ ID NO: 3 - DNA, 12 bp, artificial
  synthetic
gaaagtgaaact

SEQ ID NO: 4 - DNA, 12 bp, artificial
  synthetic
agtttcactttc

SEQ ID NO: 5 - DNA, 12 bp, Artificial
  synthetic
gaaagcgaaagc

SEQ ID NO: 6 - DNA, 12 bp, Artificial
  synthetic
gaaatcgaaagc

SEQ ID NO: 7 - DNA, 12 bp, Artificial
  synthetic
gaaagcgaaatc

SEQ ID NO: 8 - DNA, 11 bp, Artificial
  synthetic
gaaacgaaagc

SEQ ID NO: 9 - DNA, 12 bp, Artificial
  synthetic
gaaagcgaaatt

SEQ ID NO: 10 - DNA, 11 bp, Artificial
  synthetic
gaaatagaaac

SEQ ID NO: 11 - DNA, 12 bp, Artificial
  synthetic
gaaactgaaagt

SEQ ID NO: 12 - DNA, 11 bp, Artificial
  synthetic
gaaaaagaaac

SEQ ID NO: 13 - DNA, 12 bp, Artificial
  synthetic
gaaaatgaaact

SEQ ID NO: 14 - DNA, 12 bp, Artificial
  synthetic
gaaaatgaaact

SEQ ID NO: 15 - DNA, 11 bp, Artificial
  synthetic
gaaatagaaac

SEQ ID NO: 16 - DNA, 12 bp, Artificial
  synthetic
gaaactgaaagt

SEQ ID NO: 17 - DNA, 11 bp, Artificial
  synthetic
gaaatgaaagc

SEQ ID NO: 18 - DNA, 11 bp, Artificial
  synthetic
gaaacgaaagc

SEQ ID NO: 19 - DNA, 12 bp, Artificial
  synthetic
gaaactgaaaat

SEQ ID NO: 20 - DNA, 12 bp, Artificial
  synthetic
gaaagtgaaact

SEQ ID NO: 21 - DNA, 12 bp, Artificial
  synthetic
gaaagtgaaacc

SEQ ID NO: 22 - DNA, 10 bp, Artificial
  synthetic
gaaaggaaac

SEQ ID NO: 23 - DNA, 11 bp, Artificial
  synthetic
gaaacgaaagc

SEQ ID NO: 24 - DNA, 11 bp, Artificial
  synthetic
gaaagagaaac

SEQ ID NO: 25 - DNA, 10 bp, Artificial
  synthetic
gaaacgaaac

SEQ ID NO: 26 - DNA, 10 bp, Artificial
  synthetic
gaaatgaaac

SEQ ID NO: 27 - DNA, 12 bp, Artificial
  synthetic
gaaagcgaaagc

SEQ ID NO: 28 - DNA, 12 bp, Artificial
  synthetic
gaaagtgaaatt

SEQ ID NO: 29 - DNA, 12 bp, Artificial
  synthetic
gaaaacgaaact

SEQ ID NO: 30 - DNA, 10 bp, Artificial
  synthetic
gaaaggaaac

SEQ ID NO: 31 - DNA, 10 bp, Artificial
  synthetic
gaaaggaaac

SEQ ID NO: 32 - DNA, 12 bp, Artificial
  synthetic
gaaagcgaaagc

SEQ ID NO: 33 - DNA, 12 bp, Artificial
  synthetic
gaaaccgaaact

SEQ ID NO: 34 - DNA, 12 bp, Artificial
  synthetic
gaaatcgaaact

SEQ ID NO: 35 - DNA, 12 bp, Artificial
  synthetic
gaaactgaaact

SEQ ID NO: 36 - DNA, 12 bp, Artificial
  synthetic
gaaagtgaaatt

SEQ ID NO: 37 - DNA, 12 bp, Artificial
  synthetic
gaaagcgaaact

SEQ ID NO: 38 - DNA, 11 bp, Artificial
  synthetic
gaaatgaaagc

SEQ ID NO: 39 - DNA, 11 bp, Artificial
  synthetic; wherein n is a, c, g or t, and wherein g at position 10 may be absent.
gaaangaaagc

SEQ ID NO: 40 - DNA, 12 bp, Artificial
  synthetic; wherein n is a, c, g or t, and wherein g at position 11 may be absent.
gaaanngaaagc

SEQ ID NO: 41 - DNA, 12 bp, homo sapiens
agaaaagaaact

SEQ ID NO: 42 - DNA, 12 bp, homo sapiens
ggaaacgaaacc

SEQ ID NO: 43 - DNA, 13 bp, homo sapiens
ggaaactgaaact

SEQ ID NO: 44 - DNA, 13 bp, homo sapiens
ggaaaatgaaact

SEQ ID NO: 45 - DNA, 13 bp, homo sapiens
ggaaactgaaact

SEQ ID NO: 46 - DNA, 13 bp, homo sapiens
agaaactgaaaca

SEQ ID NO: 47 - DNA, 13 bp, homo sapiens
ggaaagtgaaact

SEQ ID NO: 48 - DNA, 13 bp, homo sapiens
ggaaaccgaaact

SEQ ID NO: 49 - DNA, 13 bp, homo sapiens
ggaaagtgaaacc

SEQ ID NO: 50 - DNA, 13 bp, homo sapiens
ggaaacagaaact

SEQ ID NO: 51 - DNA, 13 bp, homo sapiens
agaaatggaaagt

SEQ ID NO: 52 - DNA, 13 bp, homo sapiens
ggaaaacgaaact

SEQ ID NO: 53 - DNA, 12 bp, homo sapiens
ggaaacgaaagc

SEQ ID NO: 54 - DNA, 13 bp, homo sapiens
ggaaactgaaacc

SEQ ID NO: 55 - DNA, 12 bp, homo sapiens
agaaacgaaacc

SEQ ID NO: 56 - DNA, 13 bp, mus
ggaaaacgaaaca

SEQ ID NO: 57 - DNA, 13 bp, mus
ggaaattgaaagc

SEQ ID NO: 58 - DNA, 12 bp, mus
agaaacgaaact

SEQ ID NO: 59 - DNA, 12 bp, gallus gallus
agaaacgaaact

SEQ ID NO: 60 - DNA, 13 bp, onchorynchus mykiss
tgaaagtgaaact

SEQ ID NO: 61 - DNA, 13 bp, onchorynchus mykiss
tgaaagtgaaaca

SEQ ID NO: 62 - DNA, 12 bp, artificial
  synthetic, wherein n is a, c, g or t or may be absent
ngaaactnnnnn

SEQ ID NO: 63 - DNA, 12 bp, artificial
  synthetic
rgaaangaaasy

SEQ ID NO: 64 - DNA, 13 bp, artificial
  synthetic
rgaaanngaaasy

SEQ ID NO: 65 - DNA, 13 bp, artificial
  synthetic
ngaaanngaaact

SEQ ID NO: 66 - DNA, 12 bp, artificial
  synthetic
dgaaangaaash

SEQ ID NO: 67 - DNA, 13 bp, artificial
  synthetic
dgaaanngaaash